(19) United States
(12) Patent Application Publication
(10) Pub. No.: US 2023/0190619 A1
(43) Pub. Date: Jun. 22, 2023

(54) COSMETIC COMPOSITION COMPRISING VITAMIN B3 COMPOUND AND LAUROYL LYSINE

(71) Applicant: The Procter & Gamble Company, Cincinnati, OH (US)

(72) Inventor: Shuhei Tanaka, Singapore (SG)

(21) Appl. No.: 18/081,915

(22) Filed: Dec. 15, 2022

Related U.S. Application Data
(60) Provisional application No. 63/415,648, filed on Oct. 12, 2022.
(60) Provisional application No. 63/415,645, filed on Oct. 12, 2022.
(60) Provisional application No. 63/415,643, filed on Oct. 12, 2022.
(60) Provisional application No. 63/352,643, filed on Jun. 16, 2022.
(60) Provisional application No. 63/291,949, filed on Dec. 21, 2021.
(60) Provisional application No. 63/352,642, filed on Jun. 16, 2022.
(60) Provisional application No. 63/291,948, filed on Dec. 21, 2021.
(60) Provisional application No. 63/423,052, filed on Nov. 7, 2022.

(51) Int. Cl. A61K 8/67; A61K 8/44

(52) U.S. Cl. CPC A61K 8/675; A61K 8/44; A61K 2800/48; A61K 2800/412

(57) ABSTRACT

A cosmetic composition comprising containing from about 1% to about 15% of a vitamin B3 compound, from about 0.1% to about 10% lauroyl lysine, and from about 55% to about 98% water. The cosmetic composition can meet at least one of the following conditions: (i) the weight ratio between the vitamin B3 compound and the lauroyl lysine is from about 15:1 to about 1:10; (ii) the lauroyl lysine has a mean particle size of from about 10 microns to about 40 microns; and (iii) the lauroyl lysine has a flat polygonal shape. The cosmetic composition with vitamin B3 can provide benefits from vitamin B3 with reduced friction, reduced draggy feeling and/or improved smooth feeling.

### FIELD OF THE INVENTION

The present invention is directed to a cosmetic composition comprising by weight: from about 1% to about 15% of vitamin B₃ compound; from about 0.1% to about 10% of lauroyl lysine; and from about 55% to about 98% of water, wherein the composition meets at least one of the following conditions (i)-(iii): (i) wherein the weight ratio between the vitamin B₃ compound and lauroyl lysine is from about 15:1 to about 1:10; (ii) wherein the lauroyl lysine has a mean particle size of from about 10 microns to about 40 microns; and (iii) wherein the lauroyl lysine has a flat polygonal shape. The composition can provide benefits from vitamin B₃ compounds along with reduced friction, reduced draggy feeling and/or improved smooth feeling.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin i.e., flaking, scaling, dryness, and roughness.

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling, spots, and other histological changes typically associated with the aging of skin or environmental damage to human skin. Consumers may prefer topically applied products since they are not only effective, but also safe and pleasant to use.

Some of cosmetic products contain vitamin B₃ compounds such as niacinamide, for example, for regulating a variety of skin conditions.

However, it has been found by the inventors that, skin compositions containing vitamin B₃ compounds such as niacinamide may have higher friction, a draggy feeling and/or reduced smooth feeling during and after the application to skin, especially: when vitamin B₃ compounds are contained in cosmetic compositions containing a higher level of water; when vitamin B₃ compounds are contained in the cosmetic compositions containing lower level of humectants especially glycerin; when vitamin B₃ compounds are contained in the cosmetic compositions containing lower level of oils; and/or when vitamin B₃ compounds are contained at a higher level in such cosmetic compositions. Without being limited to a theory, but it is believed that such higher friction, draggy feeling and/or reduced smooth feeling is due to re-solidification on skin of vitamin B₃ compounds such as niacinamide during and after the application to skin.

Based on the foregoing, there is a need for a cosmetic composition, which provides benefits from vitamin B₃ compounds with reduced friction, reduced draggy feeling and/or improved smooth feeling, especially: when vitamin B₃ compounds are contained in cosmetic compositions containing higher level of water; when vitamin B₃ compounds are contained in the cosmetic compositions containing lower level of humectants especially glycerin; when vitamin B₃ compounds are contained in the cosmetic compositions containing lower level of oils; and/or when vitamin B₃ compounds are contained at a higher level in such cosmetic compositions.

Additionally, there may exists a need for a cosmetic composition, which provides at least one of the followings:
benefits from vitamin B₃ compounds with reduced friction, reduced draggy feeling and/or improved smooth feeling, while providing fresh/light feel from the composition containing a higher level of water;
benefits from vitamin B₃ compounds with reduced friction, reduced draggy feeling and/or improved smooth feeling, while providing fresh/light feel from the composition containing lower level of humectants especially glycerin;
benefits from vitamin B₃ compounds with reduced friction, reduced draggy feeling and/or improved smooth feeling, while reducing greasy gloss/oily shine from oil from the composition; and
improved benefits from vitamin B₃ compounds by a higher level of vitamin B₃ compounds, with reduced friction, reduced draggy feeling and/or improved smooth feeling.

### SUMMARY OF THE INVENTION

The present invention is directed to A cosmetic composition comprising by weight:
from about 1% to about 15% of vitamin B₃ compound;
from about 0.1% to about 10% of lauroyl lysine; and
from about 55% to about 98% of water,
wherein the composition meets at least one of the following conditions (i)-(iii):
(i) wherein the weight ratio between the vitamin B₃ compound and lauroyl lysine is from about 15:1 to about 1:10;
(ii) wherein the lauroyl lysine has a mean particle size of from about 10 microns to about 40 microns; and
(iii) wherein the lauroyl lysine has a flat polygonal shape.

It has now been surprisingly found that, by the use of lauroyl lysine in a specific condition together with vitamin B₃ compounds, the cosmetic composition can provide benefits from vitamin B₃ compounds with reduced friction, reduced draggy feeling and/or improved smooth feeling, even when vitamin B₃ compounds are contained in cosmetic compositions containing higher levels of water; when vitamin B₃ compounds are contained in the cosmetic compositions containing lower levels of humectants especially glycerin; when vitamin B₃ compounds are contained in the cosmetic compositions containing lower levels of oils; and/or when vitamin B₃ compounds are contained at a higher level in such cosmetic compositions.

### BRIEF DESCRIPTION OF THE DRAWINGS

FIG. 1 illustrates a Coefficient of friction of Comp2 (Niacinamide 5%) vs Comp3 (Niacinamide 5%+2% Amihope LL)

FIG. 2 illustrates a Coefficient of friction of Comp4 (Niacinamide 10%) vs CompS (Niacinamide 10%+2% Amihope LL)

### DETAILED DESCRIPTION OF THE INVENTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions can comprise, consist essentially of, or consist of, the essential components as well as optional ingredients described herein. As used herein, “consisting essentially of” means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+/−20%) or less (e.g., less than 15%, 10%, or even less than 5%) of the stated value.

“Apply” or “application”, as used in reference to a composition, means to apply or spread the compositions of the present invention onto a human skin surface such as the epidermis.

“Derivative,” herein, means amide, ether, ester, amino, carboxyl, acetyl, and/or alcohol derivatives of a given compound.

“Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Synergy,” and variations thereof, means that the effect provided by a combination of two or more materials is more than the additive effect expected for these materials.

“Treatment period,” as used herein, means the length of time and/or frequency that a material or composition is applied to a target skin surface.

### Cosmetic Composition

The cosmetic composition can comprise: vitamin B₃ compound; lauroyl lysine; and water, wherein the composition meets at least one of the following conditions (i)-(iii):
(i) wherein the weight ratio between the vitamin B₃ compound and lauroyl lysine is from about 15:1 to about 1:10, alternatively from about 10:1 to about 1:10, alternatively from about 8:1 to about 1:5;
(ii) wherein the lauroyl lysine has a mean particle size of from about 10 microns to about 40 microns, alternatively from about 15 microns to about 35 microns, alternatively from about 20 microns to about 30 microns; and
(iii) wherein the lauroyl lysine has a flat polygonal shape.

The composition has a viscosity of alternatively from about 100 cP to about 20,000 cP, alternatively from about 1000 cP to about 15,000 cP, especially when the composition has a higher level of water.

If the composition contains cetyl alcohol, stearyl alcohol and/or behenyl alcohol, it may be preferred that the composition contains a limited amount of such cetyl alcohol, stearyl alcohol and behenyl alcohol, for providing fresh/light feel from the composition containing a higher level of water. Total level of cetyl, stearyl, and behenyl alcohols in the composition can be up to about 0.5%, alternatively up to about 0.3%, alternatively up to about 0.1%, alternatively 0%.

If the composition contains liquid oils (at ambient) having a higher refractive index, it may be preferred that the composition contains a limited amount of such liquid oils i.e., those having a refractive index of over 1.455, for providing reduced greasy gloss/oily shine from oils from the composition. Total level of such liquid oils in the composition may be up to about 3%, alternatively up to about 1%, alternatively up to about 0.5%.

If the composition contains liquid oils (at ambient), total level of such liquid oils (at ambient) in the composition, regardless of the refractive index, alternatively up to about 10%, alternatively 7% or less. The cosmetic compositions herein are intended for topical application to human skin. The compositions herein may optionally include one or more additional skin actives or other ingredients of the type commonly included in topical cosmetic compositions.

The cosmetic compositions herein may be cosmetic compositions, pharmaceutical compositions, or cosmeceutical compositions, and may be provided in various product forms, including, but not limited to, solutions, suspensions, lotions, gels, toners, cleansing liquid washes hydrogels, film-forming products, and the like. In some instances, the composition form may follow from the particular dermatologically acceptable carrier chosen. For example, the composition (and carrier) may be provided in the form of an emulsion (e.g., water-in-oil, oil-in- water, or water-in-oil-in water) or an aqueous dispersion. The cosmetic composition can be in the form of an oil-in-water emulsion.

### Vitamin B₃ Compound

The present composition includes a safe and effective amount of a vitamin B₃ compound for regulating a variety of skin condition, for example, as described in U.S. Pat. No. 5,939,082.

The compositions herein contain from about 1% to about 15%, alternatively from about 1.5% to about 15%, alternatively from about 2% to about 15%, alternatively from about 3.5% to about 15% of vitamin B₃ compound.

As used herein, “vitamin B₃ compound” means a compound having the formula:

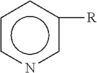

### Where:

R is CONH₂ (i.e., niacinamide), COOH (i.e., nicotinic acid) or CH₂OH (i.e., nicotinyl alcohol); derivatives thereof; and salts of any of the foregoing. Exemplary derivatives of vitamin B₃ compounds include nicotinic acid esters, including non-vasodilating esters of nicotinic acid (e.g., tocopheryl nicotinate, myristyl nicotinate) nicotinamide riboside, nicotinyl amino acids, nicotinyl alcohol esters of carboxylic acids, nicotinic acid N-oxide, and niacinamide N-oxide. In some instances, vitamin B₃ compounds such as niacinamide may have improved efficacy at lower pH, for example, as described in U.S. Publication No. 2020/0009123. Niacinamide may be used as the vitamin B₃ compound in the present invention.

In some instances, it may be desirable for the ring nitrogen of the vitamin B₃ compound to be “uncomplexed” (e.g., chemically unbound and/or unhindered) in the composition and/or prior to application to a target skin surface. For example, the compositions herein may be free of or substantially free of (i.e., less than 3%, 2%, 1% or even less than 0.5%) a salt or complex of a vitamin B₃ compound. Exemplary approaches to minimizing or preventing the formation of undesirable salts and/or complexes include omission of materials that form substantially irreversible or other undesirable complexes with the vitamin B₃ compound in the composition, pH adjustment, ionic strength adjustment, the use of surfactants, and practicing formulation processes wherein the vitamin B₃ compound and materials which complex therewith are in different phases.

### Humectant

The composition can comprise a humectant at a level of from about 0.5% to about 20%, alternatively from about 1% to about 15%, alternatively from about 2% to about 15%, alternatively form about 4% to about 12% by weight of the composition.

In the present invention, when glycerin is contained in the composition as humectant, glycerin may be included at a level such that the weight ratio between glycerin and vitamin B₃ compounds is in the scope of up to about L5:1, alternatively up to about 1.2:1, alternatively up to about 1:1, alternatively up to about 0.8:1. The glycerin can be included at a level such that the weight ratio between glycerin and vitamin B₃ compounds is in the scope of starting from about

Humectants useful herein are polyhydric alcohols intended for moisturizing, reducing scaling and stimulating removal of built-up scale from the skin. Polyhydric alcohols useful herein include, for example, alkylene polyols and their derivatives, polyalkylene glycols. Polyhydric alcohols useful herein are, for example, propylene glycol, dipropylene glycol, polypropylene glycol, polyethylene glycol, sorbitol, hydroxypropyl sorbitol, hexylene glycol, pentylene glycol, 1,3-butylene glycol, 1,2,6-hexanetriol, glycerin, ethoxylated glycerin, propoxylated glycerin and mixtures thereof. In some examples, the humectants can include glycerin, pentylene glycol, 1,3-butylene glycol, propylene glycol. The humectant can be glycerin, especially when using with Vitamin B₃ compounds at a higher level which may cause draggy feel during application and/or reduced moisturized after application.

### Lauroyl Lysine

The composition can comprise lauroyl lysine at a level of from about 0.1% to about 10%, alternatively from about 0.3% to about 7%, alternatively from about 0.5% to about 5%.

The shape of the lauroyl lysine can be anything. The shape of the lauroyl lysine can be spherical or flat polygonal (selected from flat pentagonal, flat hexagonal, and flat heptagonal, preferably flat hexagonal shape).

The lauroyl lysine in the flat polygonal shape can have a mean particle size of from about 10 microns to about 40 microns, alternatively from about 15 microns to about 35 microns, alternatively from about 20 microns to 30 microns, in view of improved sensory feeling, as too small particles may not provide enough bouncy/soft feel and too big particles may provide scrubbing-like feeling.

Commercial example of such lauroyl lysine in the flat polygonal shape having such particle size is, for example, Amihope LL available from Ajinomoto, having a flat hexagonal shape and having a mean particle size of 20-30 microns.

The lauroyl lysine can be spherical shape and can have a mean particle size of from about 1 micron to about 30 microns, alternatively from about 2 microns to about 20 microns, alternatively from about 3 microns to 8 microns.

It is believed that the lauroyl lysine provides improved moisturizing/cushioning feel, compared to starch solid powders, cellulose solid powders, and can address a potential need to improve dry and/or powdery feel during or after the application of the composition to skin which may be caused by Vitamin B₃ compounds such as niacinamide due to its re-solidification on skin.

Also, compared to hydrophilic solid polymeric powders such as starch solid powders and cellulose solid powders, it is believed that the lauroyl lysine provides reduced pilling and/or clumping especially when the composition contains higher levels of hydrophilic thickeners. For such reduced pilling and/or clumping, it may be preferred that the composition is substantially free of such hydrophilic solid powders, i.e., contains 0.1% or less, alternatively 0.05% or less of such hydrophilic solid powders. Alternatively, the composition can be free of such hydrophilic solid powders, i.e., contains 0% of such hydrophilic solid powders.

It is also believed the lauroyl lysine may provide matt appearance and/or reduced visibility of skin pores.

### Aqueous Carrier/Water

The composition can comprise an aqueous carrier. The aqueous carrier can be substantially water. Deionized water can be used. Water from natural sources including mineral cations can also be used, depending on the desired characteristic of the composition.

The composition can comprise water at a level of from about 55% to about 98%, alternatively from about 60% to about 98%, alternatively from about 60% to about 95%, alternatively from about 60% to about 90%, by weight of the composition.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils).

### pH of the Composition

Typically, cosmetic compositions are formulated to have a slightly acidic to neutral pH (i.e., 5.0-7.0), which is believed to improve the stability of certain ingredients in the composition (e.g., niacinamide, salicylates, and neutralized thickeners). However, formulating a skin care composition at low pH (e.g., 2.0-5.0) may also provide certain benefits such as improving the appearance of skin, bolstering the acid mantle of the skin, exfoliating the skin, improving skin texture, and/or providing flexibility in product formulation.

The composition may have a lower pH, i.e., pH of less than 5.0, alternatively from about 1.5 to about 4.9, alternatively from about 2.0 to about 4.5, alternatively from about 2.5 to about 4.0, or alternatively from about 3.5 to about 4.0.

### Substantially Free of Microplastic Solid Particulates

The composition can contain lauroyl lysine can provide benefits from vitamin B3 compounds, where the composition can have reduced friction, reduced draggy feeling and/or improved smooth feeling, without the use of microplastic solid particulates. Thus, the composition may also provide environmental benefit in view of the reduction of the use of microplastic solid particulates. Such microplastic solid particulates are, for example, nylon powder, polyurethane powder, polyethylene powder, silicone resin powder. The composition can be substantially free of such microplastic solid particulates. The composition can be free of such microplastic solid particulates, i.e., contains 0% of such microplastic solid particulates.

### Skin Conditioning Agent

Optionally, the composition can further comprise a skin conditioning agent. These agents may be selected from humectants and emollients. The amount of skin-condition agent may range from about 1% to about 50%, alternatively from about 2% to about 40%, alternatively from about 5% to about 30%, by weight of the composition.

Humectants are polyhydric alcohols intended for moisturizing, reducing scaling and stimulating removal of built-up scale from the skin. Typical polyhydric alcohols include polyalkylene glycols and alternatively alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. The humectant can be glycerin.

When the conditioning agent is an emollient it may be selected from hydrocarbons, fatty acids, fatty alcohols and esters.

### Fatty Alcohol

The compositions herein may include a fatty alcohol. Fatty alcohols refer to high-molecular-weight, straight-chain primary alcohols that have the general structure:

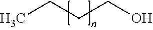

where n=8 to 32.

Fatty alcohols may be natural or synthetic, saturated or unsaturated, branched or straight-chain. Some nonlimiting examples of fatty alcohols commonly used in cosmetic compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol).

The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B 3 compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions can contain from about 0.1% to about 10%, alternatively from about 0.2% to about 5%, by weight of the composition, of a whitening agent. Ascorbic acid compounds are useful whitening agents and ascorbyl glucoside can be a preferred derivative of the ascorbic acid compounds.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Particularly suitable examples of conditioning agents include non-volatile silicone fluids, such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization.

### Hydrophilic Thickener

The compositions may further include one or more hydrophilic thickener. When present, the composition can include from about 0.01% to about 5%, alternatively from about 0.1% to about 4%, and alternatively from about 0.1% to about 3%, by weight of the composition of the hydrophilic thickeners.

The hydrophilic thickener useful herein is not particularly limited as long as it is one that is normally used in cosmetic products. Examples include natural or semi-synthetic water-soluble polymers, synthetic water-soluble polymers and inorganic water-soluble polymers.

As the natural or semi-synthetic water-soluble polymers, polysaccharides and derivatives thereof (including water-soluble alkyl-substituted polysaccharide derivatives) can be used. Specific examples include plant-based polymers such as gum arabic, tragacanth gum, galaetan, guar gum, carob gum, karaya gum, carrageenan, pectin, agar, quince seed (marmelo), algecolloid (phaeophyceae extract), starch (rice, corn, potato, wheat) and glycyrrhizinic acid; microbe-based polymers such as xanthan gum, dextran, succinoglycan and pullulan; starch-based polymers such as carboxymethyl starch and methylhydroxypropyl starch; cellulose-based polymers such as methyl cellulose, nitrocellulose, ethyl cellulose, methyl hydroxypropyl cellulose, hydroxyethyl cellulose, sodium cellulose sulfate, hydroxypropyl cellulose, sodium carboxymethyl cellulose (CMC), crystalline cellulose and cellulose powder; and alginic acid-based polymers such as sodium alginate and propylene glycol esters of alginic acid.

The synthetic water-soluble polymers include ionic or non-ionic water-soluble polymers, for example, vinyl-based polymers such as polyvinyl alcohol, polyvinyl methyl ether, polyvinyl pyrrolidone and carboxyvinyl polymers (carbomers); acryl-based polymers such as sodium polyacrylate, poly ethyl acrylate, polyacrylamide compounds and acrylic acid/alkyl methacrylate copolymers (product name “pemulen TR-1”).

The polyacrylamide compounds particularly include polyacrylamide compounds consisting of homopolymers, copolymers or crosspolymers containing one or more constituent units chosen from among 2-acrylamido-2-methylpropane sulfonic acid (hereinafter sometimes abbreviated to “AMPS”), acrylic acid and derivatives thereof.

Specific examples of such polyacrylamide compounds include vinylpyrrolidone/2-acrylamido-2-methylpropane sulfonic acid (salt) copolymers, dimethylacrylarnide/2-acrylamido-2-methylpropane sulfonic acid (salt) copolymers, acrylamide/2-acrylamiclo-2-methylpropane sulfonic acid copolymers, dimethylacrylainide/2-acrylamido-2-methylpropane sulfonic acid crosspolymers crosslinked with methylenebisacrylamide, mixtures of polyacrylamide and sodium polyacrylate, sodium acrylate/2-acrylamido-2-methylpropane sulfonic acid copolymers, hydroxyethyl acrylate/2-acrylamido-2-methylpropane sulfonic acid (salt) copolymers, ammonium polyacrylate, polyacrylamide/ammonium acrylate copolymers, and acrylamide/sodium acrylate copolymers. However, the compounds are not limited to these examples.

Examples of salts in the previous paragraph can include alkali metal salts (such as calcium salts and magnesium salts), ammonium salts, organic amine salts (such as monoethanolamine salts, diethanolamine salts, and triethanolamine salts). One or more of these polyacrylamide compounds may be used.

These polyacrylamide compounds may be synthesized or obtained as commercial products. For example, the vinyl pyrrolidone/2-acrylarnido-2-methylpropane sulfonic acid (salt) copolymer may be “Aristoflex AVC” (manufactured by Clariant), the sodium alylate/2-acrylamido-2-methylpropane sulfonic acid (salt) copolymer may be “Simulgel EG” (manufactured by Seppic) or “Simulgel EPG” (manufactured by Seppic), the acrylamide/2-acrylamido-2-methylpropane sulfonic acid sodium salt copolymer may be “Simulgel 600” (manufactured by Seppic), the acrylamide/2-acrylamido-2-methylpropane sulfonic acid (salt) may be “Sepigel 305” (manufactured by Seppic) or “Sepigel 501” (manufactured by Seppic), the homopolymer of a 2-acrylamido-2-methylpropane sulfonic acid sodium salt may be “1-lostacerin AMPS” (manufactured by Clariant) or “Simulgel 800” (manufactured by Seppic), and the dimethylacrylamide/2-acrylamido-2-methylpropane sulfonic acid may be “SU-Polymer 0-1” (manufactured by Toho Chemical Industry).

It may be preferred to use: carboxyvinyl polymers (carbomers), for example, those having tradenames of Carbopol 940 and Carbopol 980; and acrylic acid/alkyl (meth)acrylate copolymers, for example that having an INCI name of Acrylates/C10-30 Alkyl Acrylate Crosspolymer, for example, those having tradenames of Carbopol 1342 Polymer, Carbopol Ultrez 20, Carbopol Ultrez 21, Pemulen TR-1, and Pemulen TR-2. By the use of lauroyl lysine together with these thickeners, the composition provides reduced stickiness and/or draggy feel coming from these thickeners especially when using such polymers at a higher level such as 0.2% or more, while having a sufficient thickening efficacy from these thickeners. These polymers can be contained in the composition at a level of from about 0.05% to about 3%, alternatively from about 0.1% to about 2.5%, alternatively from about 0.2% to about 2%.

Polyacrylate crosspolymer-6, which is commercially available as “Sepimax Zen” from Seppic can be also used herein, especially in low pH environments.

It may be preferred to use the hydrophilic thicker together with humectants. By the use of lauroyl lysine together with hydrophilic thickeners and humectants, the composition reduces stickiness feel coming from these thickeners when used with humectant, the thickening polymer provide more sticky feel.

The hydrophilic thickener in the water-based cosmetic may be a combination of one or more types.

### Emulsifiers

When the composition is in the form of an emulsion, it may contain an emulsifier. Emulsifiers may be nonionic, anionic, cationic, or zwitterionic. Some non-limiting examples of emulsifiers are disclosed in U.S. Pat. Nos. 3,755,560, 4,421,769, U.S. Publication No. 2006/0275237 and McCutcheon's Detergents and Emulsifiers, North American Edition, pages 317-324 (1986). Suitable examples of emulsifiers include non-ionic stearic acid derivative emulsifiers such as Polysorbate-20, glyceryl stearate, sorbitan stearate, PEG-100 stearate, Steareth-2, Steareth-21, glycereth-25 pyrrolidonecarboxylic acid isostearate and combinations of these.

### Other Optional Ingredients.

Compositions herein may include one or more optional ingredients known for use in topical cosmetic compositions, provided that the optional components do not unacceptably alter the desired benefits of the composition. In some instances, it may be desirable to select cosmetic actives that function via different biological pathways so that the actives do not interfere with one another. When the composition is in the form of an emulsion, the additional ingredients should not introduce instability into the emulsion (e.g., syneresis). For example, it may be desirable to select optional ingredients that do not form complexes with other ingredients in the composition, especially pH sensitive ingredients like vitamin B3 compounds, salicylates and peptides.

The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some nonlimiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), vitamin B5 compounds (e.g., d-panthenol), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237;

2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Sucrose esters may be used herein. Such sucrose ester can be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein the sucrose dilaurate is present at 35% or more, by weight of the sucrose ester. A particularly suitable example of a sucrose ester for use herein is Sucrose Dilaurate BC10034 available from BASF.

### Method of Use of the Cosmetic Composition

The method of use herein includes identifying a target portion of skin on a person in need of treatment and applying the composition to the target portion of skin over the course of a treatment period. The target portion of skin may be on a facial skin surface such as the forehead, perioral, chin, periorbital, nose, and/or cheek) or another part of the body (e.g., hands, arms, legs, back, chest). The person in need of treatment is one whose skin exhibits signs of oxidative stress, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target such an area of skin, if it is one that typically develops such issues as a person ages. In this way, the present method may be used as a preventative measure to delay the onset of visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

### Combinations

1. A cosmetic composition comprising by weight:
from about 1% to about 15% of vitamin B₃ compound;
from about 0.1% to about 10% of lauroyl lysine; and
from about 55% to about 98% of water,
wherein the composition meets at least one of the following conditions (i)-(iii):
(i) wherein the weight ratio between the vitamin B₃ compound and lauroyl lysine is from about 15:1 to about 1:10;
(ii) wherein the lauroyl lysine has a mean particle size of from about 10 microns to about 40 microns; and
(iii) wherein the lauroyl lysine has a flat polygonal shape.
2. The composition of the preceding feature, wherein the composition comprises from about 3.5% to about 15% of vitamin B₃ compound.
3. The composition of any of the preceding features, wherein the composition comprises from about 0.3% to about 7%, alternatively from about 0.5% to about 5% of lauroyl lysine.
4. The composition of any of the preceding features, wherein the composition further comprises from about 0.5% to about 20% of humectant.
5. The composition of any of the preceding features, further comprising up to about 0.5% of a total level of cetyl, stearyl, and behenyl alcohols.
6. The composition of any of the preceding features, further comprising glycerin at a level such that the weight ratio between glycerin and vitamin B₃ compounds is up to about 1.5:1, alternatively up to about 1.2:1, alternatively up to about 1:1, alternatively up to about 0.8:1.
7. The composition of any of the preceding features, wherein the composition further comprises from about 0.2% to about 2% of a hydrophilic thickener selected from the group consisting of: carboxyvinyl polymers; acrylic acid/alkyl (meth)acrylate copolymers; and mixtures thereof.
8. The composition of any of the preceding features, wherein the weight ratio between the vitamin B₃ compound and lauroyl lysine is from about 10:1 to about 1:10, alternatively from about 8:1 to about 1:5.
9. The composition of any of the preceding features, wherein the lauroyl lysine has a mean particle size of from about 15 microns to about 35 micron, alternatively from about 20 microns to about 30 microns.
10. Use of the composition of any of the preceding features, for reduced friction, reduced draggy feeling and/or improved smooth feeling.
11. Use of the composition of any of the preceding features, for providing the reduction of the use of microplastic solid particulates, as an environmental benefit.

### EXAMPLES

### Cosmetic Compositions (Wt %)

| | Comp. 1 | Comp. 2 | Comp. 3 | Comp. 4 | Comp. 5 | Comp. 6 |
|---|---|---|---|---|---|---|
| Water Purified | q.s. to | q.s. to | q.s. to | q.s. to | q.s. to | q.s. to |
| | 100% | 100% | 100% | 100% | 100% | 100% |
| Glycerin | 1% | 1% | 1% | 1% | 1% | 1% |
| D-panthenol | 0.5% | 0.5% | 0.5% | 0.5% | 0.5% | 0.5% |
| Lauroyl Lysine | 1.5% | — | 2% | — | 2% | 2% |
| Lactic Acid | 1.8% | 1.8% | 1.8% | 1.8% | 1.8% | 1.8% |
| Sodium Lactate | 1.3% | 1.3% | 1.3% | 1.3% | 1.3% | 1.3% |
| Disodium EDTA | 0.1% | 0.1% | 0.1% | 0.1% | 0.1% | 0.1% |
| Sodium Benzoate | 0.05% | 0.05% | 0.05% | 0.05% | 0.05% | 0.05% |
| Sepimax Zen | 1.4% | 1.4% | 1.4% | 1.4% | 1.4% | 1.4% |
| (Polyacrylate | | | | | | |
| crosspolymer-6) | | | | | | |
| Niacinamide | 3.5% | 5% | 5% | 10% | 10% | 15% |

Comp. 1, 3, 5 and 6 are the inventive examples, which are suitably used as oil-in-water emulsion cosmetic composition, in a form of essence, lotion, serum and/or gel cream. Comp. 2 and 4 are comparative examples in which lauroyl lysine is not contained. The inventive examples provide reduced friction, reduced draggy feeling and/or improved smooth feeling, compared to the comparative examples, as shown below in Results.

### Method:

i-tester (TL201Tt) from Trinity lab.inc. was used for friction measurement. The 120 micron thickness of product was prepared by drawdown bar from BYK on the surface of bio skin (A4 5T #BSC from Ohken) and dried out for 24 hours before measurement.
The friction of the dried product film on the bio skin plate was measured by the polyurethan probe with fingerprint mimic patterns with 50 g vertical loading/40 mm travel distance at 50 mm/sec for 60 counts (rotations) (data was collected at 1 msec). Measurement was repeated 3 times to calculate the average.

### Results:

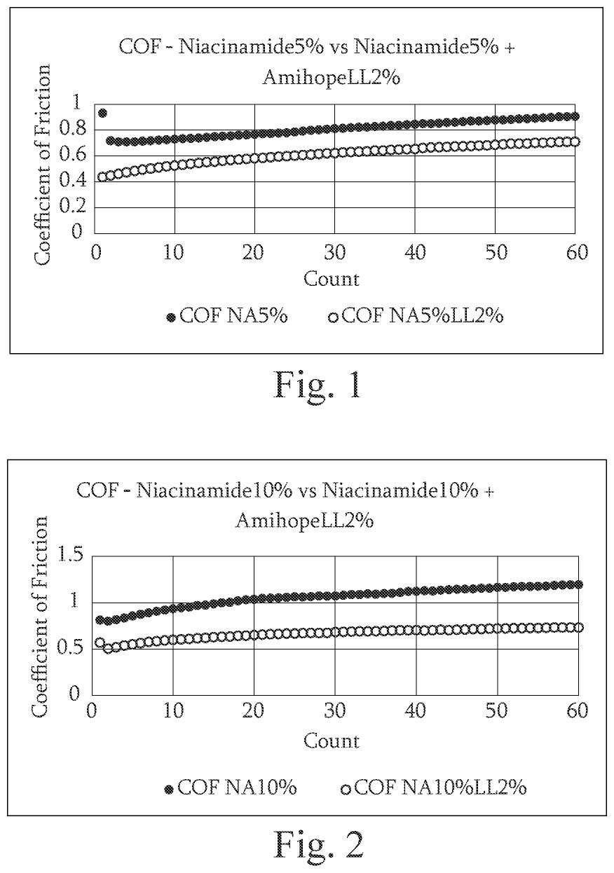

FIG. 1 shows a Coefficient of friction of Comp2 (Niacinamide 5%) vs Comp3 (Niacinamide 5%+2% Amihope LL). As FIG. 1 indicates, Comp3 formula (5% Niacinamide+2% Amihope LL) showed consistently lower Coefficient of friction (COF) than Comp2 (5% Niacinamide) during friction measurement, which indicates Comp3 formula (with Amihope LL) provides reduced friction, reduced draggy feeling and/or improved smooth feeling compared Comp2 during and after product application while containing a higher level of Niacinamide in the formula.

FIG. 2 shows a Coefficient of friction of Comp4 (Niacinamide 10%) vs CompS (Niacinamide 10%+2% Amihope LL).

As FIG. 2 indicates, CompS formula (10% Niacinamide+2% Amihope LL) showed consistently lower Coefficient of friction (COF) than Comp4 (10% Niacinamide) during friction measurement, which indicates CompS formula provides reduced friction, reduced draggy feeling and/or improved smooth feeling compared to Comp4 during and after product application, while containing a higher level of Niacinamide in the formula.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising by weight:
from about 1% to about 15% of a vitamin B₃ compound;
from about 0.1% to about 10% of a lauroyl lysine; and
from about 55% to about 98% of water;
wherein the composition meets at least one of the following conditions (i)-(iii):
(i) wherein the weight ratio between the vitamin B₃ compound and the lauroyl lysine is from about 15:1 to about 1:10;
(ii) wherein the lauroyl lysine comprises a mean particle size of from about 10 microns to about 40 microns; and
(iii) wherein the lauroyl lysine comprises a flat polygonal shape.

2. The composition of claim 1, wherein the composition comprises from about 3.5% to about 15% of the vitamin B₃ compound.

3. The composition of claim 1, wherein the composition comprises from about 0.3% to about 7% of the lauroyl lysine.

4. The composition of claim 3, wherein the composition comprises from about 0.5% to about 5% of the lauroyl lysine.

5. The composition of claim 1, wherein the composition further comprises from about 0.5% to about 20% of a humectant.

6. The composition of claim 1, further comprising up to about 0.5% of a total level of cetyl, stearyl, and behenyl alcohols.

7. The composition of claim 1, further comprising glycerin; wherein the weight ratio between the glycerin and the vitamin B₃ compound is up to about L5:1.

8. The composition of claim 7, wherein the weight ratio between glycerin and the vitamin B₃ compound is up to about 1.2:1.

9. The composition of claim 8, wherein the weight ratio between glycerin and vitamin B₃ compound is up to about 1:1.

10. The composition of claim 9, wherein the weight ratio between the glycerin and the vitamin B₃ compound is up to about 0.8:1.

11. The composition of claim 1, wherein the composition further comprises from about 0.2% to about 2% of one or more hydrophilic thickeners; wherein the one or more hydrophilic thickener comprises carboxyvinyl polymers, acrylic acid/alkyl (meth)acrylate copolymers, or a combination thereof.

12. The composition of claim 1, wherein the weight ratio between the vitamin B₃ compound and the lauroyl lysine is from about 10:1 to about 1:10.

13. The composition of claim 12, wherein the weight ratio between the vitamin B₃ compound and the lauroyl lysine is from about 8:1 to about 1:5.

14. The composition of claim 1, wherein the lauroyl lysine comprises a mean particle size of from about 15 microns to about 35 microns.

15. The composition of claim 14, wherein the mean particle size of the lauroyl lysine is from about 20 microns to about 30 microns.

16. A cosmetic composition comprising by weight:
from about 1% to about 15% of a vitamin B₃ compound
from about 0.1% to about 10% of a lauroyl lysine comprising a mean particle size of from about 10 microns to about 40 microns; and
from about 55% to about 98% of water;
wherein the weight ratio between the vitamin B₃ compound and the lauroyl lysine is from about 15:1 to about 1:10; and
wherein the lauroyl lysine comprises a flat polygonal shape.

17. The cosmetic composition of claim 16 wherein the vitamin B₃ compound comprises niacinamide.
